(19) United States
(12) Patent Application Publication
(10) Pub. No.: US 2023/0015935 A1
(43) Pub. Date: Jan. 19, 2023

(54) METHOD FOR THE ACTIVATION OR REJUVENATION OF A CATALYST

(71) Applicant: Emerging Fuels Technology, Inc., Tulsa, OK (US)

(72) Inventor: Kenneth L. Agee, Tulsa, OK (US)

(73) Assignee: Emerging Fuels Technology, Inc., Tulsa, OK (US)

(21) Appl. No.: 17/376,461

(22) Filed: Jul. 15, 2021

(51) Int. Cl. B01J 38/02; B01J 23/75; B01J 38/10; C07C 1/04

(52) U.S. Cl. CPC B01J 38/02; B01J 23/75; B01J 38/10; C07C 1/041; C07C 1/045; C07C 1/043; C07C 2523/75

(57) ABSTRACT

A method is disclosed for rejuvenation a cobalt Fischer Tropsch catalyst used in a Fischer Tropsch process operating in recycle mode. The method permits the use of specific inert gases to adjust the mole weight of the gas so that the recycle compressor designed for normal steady state operation can also be used in the method. Hydrogen from a membrane permeate stream is added to the reactor loop at a temperature between 300 F and 400 F and the carbon oxides are reacted out to purify the hydrogen. This stream is continuously recycled and the temperature is raised to between 425 F and 500 F and held at the final temperature for between 4 hours and 48 hours. The cobalt Fischer Tropsch catalyst is effectively rejuvenated in-situ by the method.

### BACKGROUND OF THE INVENTION

### Cross Reference

Not Applicable.

### Field of the Invention

This invention relates generally to a method to activate or rejuvenate a cobalt Fischer Tropsch catalyst used for the production of heavy hydrocarbon products.

### Description of the Related Art

Various processes are known for the conversion of light methane-containing gases into normally liquid products such as methanol, higher alcohols, and hydrocarbon fuels and chemicals, particularly paraffinic hydrocarbons. Such processes are directed at the objective of adding value to the light gaseous feedstock by making transportable, more valuable products such as diesel fuel or jet fuel.

The Fischer Tropsch process can be used to convert such gaseous light hydrocarbon products into more valuable, easily transportable liquid hydrocarbon products. The gaseous feedstock is first converted to synthesis gas comprising carbon monoxide and hydrogen. The synthesis gas is then converted to heavy hydrocarbon products with a Fischer Tropsch catalyst. Likewise various carbonaceous feeds can be converted to synthesis gas and such synthesis gas can be converted to heavy hydrocarbon products with a Fischer Tropsch catalyst. The heavy hydrocarbon products can be subjected to further workup by hydroprocessing such as hydrocracking and/or hydroisomerization and distillation, resulting in a high yield of high-quality middle distillate products such as jet fuel or diesel fuel. The heavy hydrocarbon products can also be upgraded to specialty products such as solvents, drilling fluids, waxes, or lube base oils due to the high purity of the Fischer Tropsch products.

Processes that convert light hydrocarbons or carbonaceous feeds to heavier hydrocarbon products generally have three steps: 1) conversion of light hydrocarbon feedstock or the carbonaceous feedstock to synthesis gas comprising carbon monoxide and hydrogen; 2) conversion of the synthesis gas to heavy hydrocarbons via the Fischer Tropsch reaction; and 3) hydroprocessing the heavy hydrocarbon product to one or more finished hydrocarbon products.

In the synthesis step, use is made of a synthesis catalyst such as a Fischer Tropsch catalyst. Fischer Tropsch catalyst are typically composed of iron or cobalt deposited on an inorganic oxide support such as silica, alumina, titania, or zirconia with various promoters such as ruthenium, platinum, rhenium, and other metals in a minor amount to enhance the catalyst performance. Examples of such Fischer Tropsch catalyst can be found in U.S. Pat. Nos. 9,180,436 and 9,358,526.

Fischer Tropsch catalyst are costly to manufacture and must be cost effective in the overall process economics. As such, the user must consider the total catalyst life, to amortize the cost of the catalyst over a large volume of products so that the cost of catalyst per unit of product is acceptable. Any method that recovers loss of catalyst activity and extends the catalyst useful life improves the amortized cost of the catalyst. The Fischer Tropsch catalyst typically has a stable but constantly decreasing rate of activity which must be managed by increasing temperature to maintain stable activity over a practical temperature range. The operator of a Fischer Tropsch process may offset loss of activity by raising temperature, but as temperature increases the amount of undesirable products (typically light hydrocarbons, i.e. C1-C4) causes a reduction in desirable products (C5+) and the process must be stopped periodically to either regenerate or replace the catalyst. Because the catalyst is expensive and the operator has the need to get the maximum yield of products from a single charge of catalyst, it is necessary to regenerate the catalyst if possible and as many times as possible. The initial activation of the catalyst is also critical as it sets the life and activity of the first operating cycle of the catalyst and conditions the catalyst for future cycles of regeneration.

Catalyst regeneration can be thought of as a way to clean and restructure the surface of the catalyst, to reverse sintering, remove carbon deposits and return it, as much as possible, to the condition of fresh catalyst. Various methods known to one skilled in the art have been used to regenerate a Fischer Tropsch catalyst. For example, one may treat the catalyst with a reducing gas such as hydrogen or one may employ a solvent to remove heavy hydrocarbons, or an oxidizing gas may be employed to burn off carbon deposits and potentially redistribute the active cobalt crystals. A combination of reducing and oxidizing such as a ROR (reduction oxidation reduction) method may be employed as well. An example of a ROR method can be found in U.S. Pat. No. 10,434,506. A simple hydrogen treatment is defined herein as a rejuvenation method. Many rejuvenation examples exist such as for example US patents assigned to ExxonMobil including U.S. Pat. Nos. 6,541,525 6,006,670 5,973,012 5,958,986 5,929,126 5,821,270 5,817,701 5,811,468 5,811,363 5,283,216, and 5,268,344. These patents describe multiple methods involving addition of special equipment internal or external to a slurry reactor to isolate a portion of the cobalt Fischer Tropsch catalyst and rejuvenate it with a hydrogen containing gas. While the method of the present invention can be used for any reactor type including a slurry reactor, it is intended for a fixed bed reactor and unlike the aforementioned patents assigned to ExxonMobil, does not require the addition of new equipment and rejuvenates the entire catalyst contents of the reactor at once. Such a method may or may not be as effective as some regeneration methods depending on how the catalyst was deactivated.

A long-term catalyst maintenance strategy may effectively use multiple rejuvenation treatments between more aggressive ROR treatments. Such ROR treatments while effective for severely deactivated catalyst may contribute to long term permanent loss of active sites. It is believed that using the rejuvenation method of the present invention in combination with ROR treatments will result in a longer total effective catalyst life and thus lower amortized catalyst cost. Heretofore, a simple hydrogen rejuvenation method suffered several drawbacks including the need for high purity hydrogen in a remote location and the process conditions require special equipment different from what is available for daily operations. One objective of the present invention is to provide a rejuvenation method that overcomes these problems.

A simple single step hydrogen treatment herein referred to as a rejuvenation for a full charge of catalyst could be difficult and expensive to practice. Such a process involves exposure of the catalyst to a high purity hydrogen stream at elevated temperature to clean the pores and the surface of the catalyst. In a remote location, high purity hydrogen will likely not be available and would be expensive to generate requiring special equipment to generate, store, or both. Another problem is that the typical recycle compressor used for normal operation of a plant is not designed for 100% hydrogen due to the low molecular weight of hydrogen.

For these reasons and many more it is an objective of the present invention to provide a method to quickly and efficiently rejuvenate activity of a partially deactivated cobalt Fischer Tropsch catalyst with equipment and resources available at a remote plant site. A preferred embodiment of the present invention will use the existing recycle compressor of the Fischer Tropsch reactor loop. The method must also make use of a less than pure hydrogen stream such as the permeate stream of a hydrogen membrane typically part of a plant and used to adjust the H2:CO ratio of the synthesis gas before introduction into the Fischer Tropsch reactor loop. Such gas may comprise from 3% to 10% or more of carbon oxides including carbon monoxide and carbon dioxide. The Fischer Tropsch reactor loop is defined as the equipment in the gas flow path including any feed preheat or feed/effluent exchanger, the Fischer Tropsch reactor, separators such as hot and cold separators, coolers, and a recycle compressor. Membrane hydrogen may be further purified by a PSA (Pressure Swing Adsorption) treatment if available on site but such treatment is not required by the method. The rejuvenation method of the present invention has been demonstrated to effectively rejuvenate a partially deactivated cobalt Fischer Tropsch catalyst, using a hydrogen containing stream comprising substantial amounts of carbon oxides. While the method is primarily described as a rejuvenation method it may also be effective for initial catalyst activation.

Based on the foregoing, it is desirable to use the rejuvenation method to recover activity of a partially deactivated cobalt Fischer Tropsch catalyst with minimal additional equipment and cost. For a Fischer Tropsch reactor that is designed to operate in a recycle mode, it is therefore desirable to use the existing equipment such as the recycle compressor, hydrogen membrane, and product knockout drums to regenerate the catalyst.

If the Fischer Tropsch reactor is designed to operate on a once thru basis without recycle, the rejuvenation may still be operated in a recycle mode. In this case a recycle compressor may be provided on a temporary basis for the purpose of the rejuvenation. For the purpose of the present invention the reactor may be considered as operating in a recycle mode if recycle is used only for rejuvenation. If the process is designed wherein recycle is used, it is also desirable to use inert gases such as a methane containing gas or other light hydrocarbon gases or argon to dilute the hydrogen containing gas as provided herein. These hydrocarbon gases include the common feed gases used in a gas-to-liquids process such as methane, natural gas, associated gas, coal seam gas, landfill gas, or biogas. When biogas is used as the diluent gas it is preferable to remove a substantial portion of the carbon dioxide which can be done by any method known to one skilled in the art including using a CO2 selective membrane. For all feed gases care should be taken to de-sulfurize the gas before use in the method.

### SUMMARY OF THE INVENTION

In general, in a first aspect, the invention relates to a method to rejuvenate a partially deactivated cobalt Fischer Tropsch catalyst used in a synthesis process operating the Fischer Tropsch reaction in recycle mode using a recycle compressor. The method may comprise conducting a hydrogen treatment of the cobalt Fischer Tropsch catalyst at elevated pressure near the normal operating pressure of the cobalt Fischer Tropsch catalyst, where the rejuvenation may comprise circulation of a hydrogen containing gas with one or more diluent gases. Normal pressure for cobalt Fischer Tropsch catalyst is defined as 10 to 60 bar. The rejuvenation method will work below 10 bar but use of the process recycle compressor generally leads to higher pressure in order to keep the flow rate sufficiently high, on the order of normal flow for the process. For example, the flow rate may preferably be above 500 GHSV, more preferably above 1,000 GHSV. When the recycle compressor of the process is a centrifugal compressor, its design limits for mole weight and/or hydrogen concentration must be considered. Adding one or more diluent gases to the recycle loop during the process, thereby adjusting the mole weight and/or hydrogen concentration of the blended gas, may satisfy the design limits of the recycle compressor. It has surprisingly been found that with substantial diluent concentration, i.e. from 20% to 50% diluent, the rejuvenation process of the present invention is still effective, possibly because in spite of the reduced hydrogen concentration, the method is intentionally performed at an elevated pressure above 10 bar, preferably above 20 bar such that even dilute hydrogen has adequate partial pressure to be effective for the method.

The one or more diluent gases may comprise argon, and feed gases such as light saturated hydrocarbon gases including methane, natural gas, associated gas, coal seam gas, landfill gas, biogas, or a combination thereof added during the rejuvenation process. When using landfill gas or biogas it is preferred to remove a substantial portion of the CO2. The one or more diluent gases added during the process of the present invention may explicitly exclude nitrogen and carbon dioxide.

The process may use a feed gas, which may be methane, natural gas, biogas, landfill gas, coal seam gas, or associated gas. The recycle loop may have a flow rate during the rejuvenation process that is as high, or higher than the flow rate of synthesis gas during normal synthesis operation, preferable above 500 GHSV, more preferably above 1,000 GHSV. The recycle compressor may be a centrifugal compressor.

The addition of one or more diluent gases added during the rejuvenation process may avoid material changes to the recycle compressor. The rejuvenation process of the present invention may be conducted at a temperature between 300 F and 500 F. The process may require a low temperature hydrogen purification step and a higher temperature catalyst rejuvenation step. The preferred hydrogen source may be a permeate stream taken from a hydrogen membrane, which may remove hydrogen from the synthesis gas that feeds the Fischer Tropsch loop to adjust the H2:CO ratio. During rejuvenation, excess synthesis gas may be sent to a flare. The membrane permeate stream may have a high concentration of hydrogen from 90% to 95% and typically includes 3% to 10% or more of carbon oxides including carbon monoxide and carbon dioxide and some methane. These carbon sources may dilute the purity of the hydrogen that will be used in the method. If this stream is introduced into the reactor as is, it will react aggressively with the catalyst to make methane. The exothermic nature of this reaction could cause damage at high temperature by coking the surface of the catalyst that is intended to be rejuvenated. To avoid any damage to the catalyst, the loop may be initially filled with a diluent gas such as methane or natural gas. Such gases are not reactive at the temperatures of the method and have a molecular weight high enough when combined with the hydrogen rich steam to satisfy the mole weight limits of the recycle compressor. This gas may be circulated through the reactor and the loop including all separators and piping in the loop. A small amount of the low purity hydrogen (membrane permeate) may be added to the loop at a temperature between 300 F and 400 F. Carbon oxides in the hydrogen stream may react to make methane and higher hydrocarbons, which acts as an inert gas in the rejuvenation process. Additional hydrogen rich gas can be added to the recycle loop until the desired hydrogen concentration is achieved. After substantially all the carbon oxides are reacted, the resulting hydrogen and diluent gas mixture may be used to rejuvenate the partially deactivated catalyst. If a PSA system is available on site, the PSA purified hydrogen may be used and the low temperature step to remove carbon oxides may be shorter and less critical. A hydrogen concentration of anywhere between 1% and 99% can be used to rejuvenate the catalyst. A more preferred concentration is between 10% and 80% hydrogen. A still more preferred concentration is between 20% and 50% hydrogen. If it is desired to increase the hydrogen concentration, or if the hydrogen concentration decreases, additional hydrogen from the membrane can be added to the loop at the low temperature range. This hydrogen containing minor amounts of carbon oxides may be cycled in the loop at a temperature between 300 F and 400 F until most, preferably 90% or more, of the carbon oxides are converted to methane or higher hydrocarbons. For this purpose, the loop may be operated with very little or no purge. After the carbon oxides are essentially completely converted, that is at least 90% converted, the temperature of the catalyst bed may be raised to between 425 F and 500 F and held for at least 4 hours, up to 48 hours. For partially deactivated cobalt Fischer Tropsch catalyst, this rejuvenation process may recover a substantial portion of the catalyst activity.

### DETAILED DESCRIPTION OF THE INVENTION

The methods discussed herein are merely illustrative of specific manners in which to make and use this invention and are not to be interpreted as limiting in scope.

While the method has been described with a certain degree of particularity, it is to be noted that many modifications may be made in the details of the construction and the arrangement of the devices and components without departing from the spirit and scope of this disclosure. It is understood that the method is not limited to the embodiments set forth herein for purposes of exemplification.

In general, in a first aspect, the invention relates to a method to rejuvenate a cobalt Fischer Tropsch catalyst used for the production of heavy hydrocarbon products from synthesis gas.

As noted above, it is an object of the present invention to use a rejuvenation method to recover activity of a partially deactivated cobalt Fischer Tropsch catalyst with minimal additional equipment and cost. For a Fischer Tropsch reactor that is designed to operate in a recycle mode, it is therefore desirable to use the existing equipment such as the recycle compressor, hydrogen membrane, and product knockout drums to rejuvenate the catalyst. Since the Fischer Tropsch recycle process may be designed for a relatively low pressure drop and high flow, a centrifugal compressor is a likely choice for the system. Centrifugal compressors must be designed for the operating pressure and gas conditions. While a centrifugal compressor is the preferred choice of the present invention, any type of compressor known to one skilled in the art may be used and still be within the scope of the invention.

To keep the method simple and the cost low, operating conditions of the rejuvenation method will preferably be set so that the same compressor used in normal operation of the Fischer Tropsch loop can be used for the rejuvenation method. Normally the recycle compressor will operate with a mole weight in the range of approximately 10 to 30. The recycle gas in normal Fischer Tropsch operation may consist of unreacted hydrogen and carbon monoxide and system inert gases, which may include nitrogen, carbon dioxide, and light hydrocarbons. If the feed gas being processed is a biogas, it may contain large amounts of carbon dioxide, which will make the recycle gas stream mole weight higher. If the feed gas is natural gas, methane will build up as the inert gas and the mole weight will be lower. In all cases, the mole weight of the normal recycle stream is much higher than that of hydrogen, which is the gas needed in the rejuvenation method. Since hydrogen has a mole weight of 2, it will not be possible to use the existing process recycle compressor to operate the rejuvenation method without substantially changing the composition of the gas. An inert gas must be added to the hydrogen used in the rejuvenation method to increase the mole weight of the gas so that the process compressor can be used for both operations. Without the addition of an inert gas, the only other options are to provide a separate compressor for recycle or operate the method on a once-through basis, which is not practical. While the method can be practiced with a very high concentration of hydrogen, it is desirable to dilute the hydrogen. This dilute system still has adequate hydrogen since the method is operated above 10 bar, preferably above 20 bar, up to the normal operating pressure of the Fischer Tropsch reaction, which could be up to 60 bar or more. At these pressures, the hydrogen partial pressure is high and it will therefore be adequate for the rejuvenation method. In a preferred embodiment of the process the hydrogen is diluted to between 20% and 50% hydrogen concentration. By definition this is an in-situ rejuvenation, as the catalyst is fully rejuvenated in the reactor and returned to service with minimal downtime.

### Experimental

A long continuous run was carried out to demonstrate the effectiveness of the rejuvenation method of the present invention for recovering activity of a partially deactivated cobalt Fischer Tropsch catalyst. For the run a ½ inch internal diameter micro reactor was packed with a mixture of 22.5 cc of inert ceramic material and 7.5 cc of cobalt Fischer Tropsch catalyst made according to U.S. Pat. No. 9,358,526. The catalyst was initially activated using a ROR activation process described in U.S. Pat. No. 10,434,506. The experiments were done using bottled syngas with a H2:CO ratio of 1.6 and containing approximately 34% nitrogen as a diluent and internal standard for analysis. Operating conditions are listed in Table 1 below. The catalyst relative performance is described using a kinetic factor Kt. The initial Kt of the catalyst was around 3.0, while the Kt was 2.77 at 50 hours on stream and 2.45 at 150 hours on stream. After 2,200 hours of steady operation, the catalyst Kt was down to 1.54 or about 55% of its fresh 50 hour rate.

After 2,200 hours of operation, the reaction was stopped and a rejuvenation was performed according to the present invention. A hydrogen containing stream comprising 95% hydrogen and a 5% mix of CO and CO2 in roughly equivalent amounts was diluted with 50% methane and passed through the catalyst at temperatures between 300 F and 400 F for several hours, demonstrating substantial conversion of both the CO and CO2. After this, the hydrogen stream was diluted by adding methane to the hydrogen stream to a concentration of 75% methane and 25% hydrogen. The temperature was raised to 475F and held for 24 hours. The catalyst was put back on stream at conditions substantially equivalent to run 1. This operation is referred to in Table 1 as run 2 and demonstrates essentially full recovery of activity.

TABLE 1
Fischer Tropsch catalyst performance
before and after rejuvenation

| Run | Tempera- | Pressure | Flow | Hours on | Activity |
|---|---|---|---|---|---|
| number | ture (F.) | (psig) | (GHSV) | stream (Hr) | Kt |
| Run 1 | 380 | 500 | 4000 | 50 | 2.77 |
| Run 1 | 380 | 500 | 3250 | 150 | 2.45 |
| Run 1 | 380 | 500 | 2750 | 2200 | 1.54 |
| Run 2 | 365 | 500 | 2750 | 50 | 2.45 |
| Run 2 | 375 | 500 | 2750 | 150 | 2.49 |

It is not required that the flowrate of the compressor during a rejuvenation operation be the same as the flowrate in the normal Fischer Tropsch synthesis mode. It is preferable that the flowrate be equal to or higher than the flow during normal synthesis operation. The pressure drop will naturally go down in the rejuvenation mode since no liquid products are being produced during the operation of the rejuvenation method which are normally produced in the synthesis mode. Addition of one or more inert gases selected from argon, and one or more feed gases, or combinations can be used to set the mole weight of the gases used in the method of the present invention to stay within the limits of the recycle compressor designed for the normal Fischer Tropsch synthesis operation. If light hydrocarbons heavier than propane are used, they may have to be heated to keep them in the vapor phase, which can become self-limiting.

The present invention is directed at a method to rejuvenate a cobalt Fischer Tropsch catalyst used in a Fischer Tropsch process comprising:
A) purging the Fischer Tropsch reactor loop with a feed gas, argon, or a mixture thereof;
B) adding permeate hydrogen from a hydrogen membrane to the Fischer Tropsch reactor loop to between 10% and 80%, preferably from 20% to 50%, while recycling the loop with little or no purge, at a temperature below 300 F in the Fischer Tropsch reactor;
C) raising the temperature in the Fischer Tropsch reactor to between 300 F and 400 F to hydrogenate carbon oxides from the permeate gas, thus making methane and other heavier hydrocarbons, resulting in a circulating gas comprising hydrogen and relatively inert hydrocarbon gases or argon, that is substantially depleted of carbon oxides;
D) raising the temperature of the Fischer Tropsch reactor to between 425 F and 500 F and holding for between 4 hours and 48 hours while recycling the hydrogen containing gas with little or no purge.

When the rejuvenation method is completed, the temperature may be reduced to a safe operating temperature to start the Fischer Tropsch process.

Whereas, the devices and methods have been described in relation to the claims, it should be understood that other and further modifications, apart from those shown or suggested herein, may be made within the spirit and scope of this invention.

What is claimed is:

1. A method to activate or rejuvenate a cobalt Fischer Tropsch catalyst used in a Fischer Tropsch process, the method comprising:
A) purging a Fischer Tropsch reactor loop with a feed gas, argon, or a mixture thereof;
B) adding permeate hydrogen from a hydrogen membrane to the Fischer Tropsch reactor loop to between 10% and 80% while recycling the loop with little or no purge, at a temperature below 300 F in the Fischer Tropsch reactor;
C) raising the temperature in the Fischer Tropsch reactor to between 300 F and 400 F to hydrogenate carbon oxides from the permeate hydrogen, thus making methane and other heavier hydrocarbons, resulting in a circulating gas comprising hydrogen and relatively inert hydrocarbon gases or argon, that is substantially depleted of carbon oxides; and
D) raising the temperature of the Fischer Tropsch reactor to between 425 F and 500 F and holding for between 4 hours and 48 hours while recycling the hydrogen-containing gas with little or no purge.

2. The method of claim 1 wherein the feed gas is methane, natural gas, associated gas, coal seam gas, landfill gas, biogas, or a combination thereof.

4. The method of claim 1 wherein the Fischer Tropsch reactor loop comprises a recycle compressor and where the recycle compressor of the Fischer Tropsch reactor loop is a driver for recycling the gases.

5. The method of claim 1 wherein the Fischer Tropsch reactor is a fixed bed reactor.

6. The method of claim 1 wherein the method occurs at an elevated pressure above 10 bar.

7. The method of claim 1 wherein the rejuvenation occurs at an elevated pressure above 20 bar.

8. The method of claim 1 wherein the method operates at a gas flow rate equal to or greater than the flow rate used during normal synthesis operation.

9. The method of claim 1 wherein the method operates at a gas flow rate above 500 GHSV.

10. The method of claim 1 wherein the method operates at a gas flow rate above 1,000 GHSV.

11. The method of claim 1 wherein the gas is circulated with a compressor.

12. The method of claim 11 where the compressor is a centrifugal compressor.

13. The method of claim 1 wherein 90% or more of the carbon oxides are converted to methane or higher hydrocarbons in step C.

14. The method of claim 1 wherein the cobalt catalyst is rejuvenated in-situ.

15. The method of claim 1 wherein the hydrogen of step (B) is from a PSA system.
